(19) United States
(12) Patent Application Publication
(10) Pub. No.: US 2026/0174574 A1
(43) Pub. Date: Jun. 25, 2026

(54) WEIGHTLIFTING DEVICE THAT GRIPS WEIGHT IN LIEU OF A HAND

(71) Applicant: Brandon Alexander Carlton, Yorba Linda, CA (US)

(72) Inventor: Brandon Alexander Carlton, Yorba Linda, CA (US)

(21) Appl. No.: 19/000,684

(22) Filed: Dec. 23, 2024

(51) Int. Cl. A61F 4/00; A63B 21/00; A63B 21/065

(52) U.S. Cl. CPC A61F 4/00; A63B 21/065; A63B 21/4021

(57) ABSTRACT

A weightlifting device with a customized forearm brace, including elements of the customization of the device is provided. The device effectively grips a weight in lieu of a hand, thereby enabling people with upper extremity (hand, wrist, or finger) injuries, deformities, amputations and any other related afflictions to circumnavigate their ailment(s) and engage in weightlifting exercise.

### FIELD

The present application relates to gripping aid devices in the context of weightlifting exercise, however the device which is the subject of this application effectively grips a weight in lieu of a hand and does not require the use of a hand or the existence of a hand to grip a weight.

### BACKGROUND

People with upper extremity (wrist, hand, finger) injuries, deformities, or amputations are unable to effectively hold weights to weightlift. The inability to lift weights results in physical problems such as decreased muscle mass and lower bone density as well as mental and emotional problems such as depression and anxiety. Athletes lose strength, endurance, size, and progress in general when unable to lift weights. It is awkward, disproportionate, and antithetical to muscular symmetry to continue to lift weights with the unaffected parts of the body while excluding the affected hand. Existing gripping aid devices used in the context of weightlifting require the use of a hand to hold a weight. People with upper extremity injuries are unable to use their injured hand to hold a weight due to issues such as pain and splints/casts. People with upper extremity deformities such as cerebral palsy are physically incapable of using their affected hand to hold a weight. People with hand amputations do not possess a hand in which to hold a weight.

### SUMMARY

By providing a device in the form of a customized forearm brace that effectively holds and grips a dumbbell weight in lieu of a hand as well as clips onto a cable machine carabiner, people with upper extremity injuries, deformities, or amputations can circumnavigate their ailment and lift weights.

The disclosed exemplary embodiment provides a device that uses a forearm brace fitted with customized hooks and straps to securely fasten and effectively hold a dumbbell weight. A ring attached to the device enables the user to clip onto a cable machine for lifting exercises as an alternative to dumbbells.

Further objects, features, advantages, and properties of device according to the present application will become apparent from the detailed description.

### BRIEF DESCRIPTION OF THE DRAWINGS

In the following detailed portion of the present description, the teachings of the present application will be explained in more detail with reference to the example embodiment shown in the drawings, in which:

FIG. 1 is a perspective view of a device according to an embodiment from a supinated position with an exemplary human arm inserted into it and an exemplary dumbbell weight strapped to it to exhibit functionality and demonstrate overall purpose of device

FIG. 2 is a perspective view of a device according to an embodiment from a pronated position with an exemplary human arm inserted into it and an exemplary cable weight machine carabiner attached to it to exhibit functionality and demonstrate another purpose of device

FIG. 3 is a perspective view of a device according to an embodiment from a supinated position without an exemplary human arm or dumbbell weight to provide an unobstructed view of the device

FIG. 4 is an isometric view of a device according to an embodiment from a supinated position to clearly exhibit the available perspectives and features of the device from this position

FIG. 5 is an isometric view of a device according to an embodiment from a pronated position to clearly exhibit the available perspectives and features of the device from this position

FIG. 6 is a close-up partial view of a device according to an embodiment from a lateral position with an emphasis on the dumbbell strap functionality and direction of flow through tri-glide slides to exhibit the manner in which the dumbbell strap grips and secures the weight to the hooks

FIG. 7 is an exploded view of the hook system comprised of metal hooks that are connected via cross bars, nuts, and bolts to illustrate the construction of the hook system which provides the base for the dumbbell weight to rest upon

FIG. 8 is an exploded view of the layers of material used in the construction of the main part of the device, the brace, to demonstrate the order in which the materials are placed upon one another

### DETAILED DESCRIPTION

In the following detailed description, the device according to the teachings for this application in the form of a weightlifting device that grips a weight in lieu of a hand will be described by an embodiment. It should be noted that although only this embodiment and its labeled parts are described, the teachings of this application can also be used with many other variations of the embodiment including but not limited to any other arm brace device and many other fastening methods including but not limited to racks, tracks, slots, pins, knots, bolts, rivets, and clips. It should be noted that this is not an exhaustive list and there may be other variations which are not described herein.

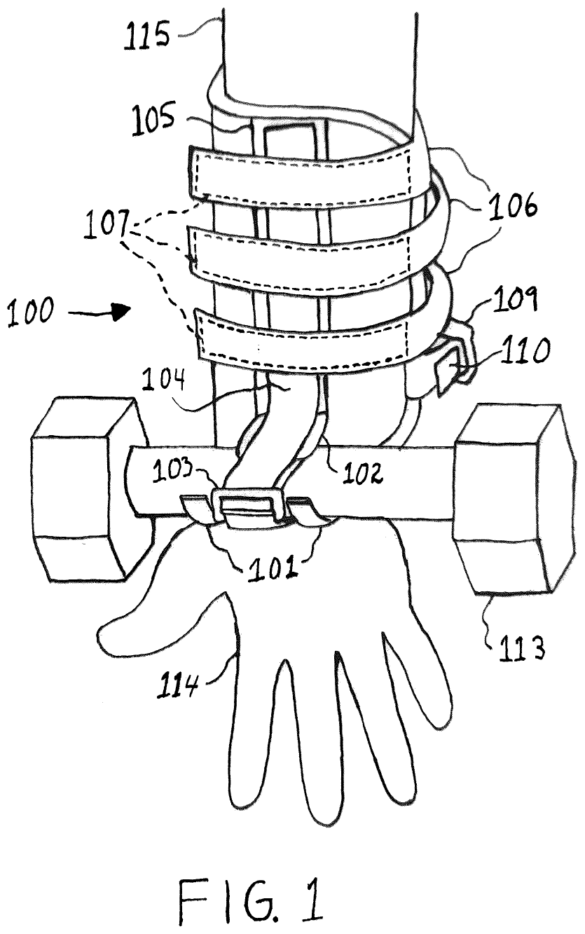

FIG. 1 illustrates the primary purpose of the device as an embodiment of brace 100 in a supinated position from a functional perspective for dumbbell weight 113 lifting. Brace 100 is worn in a similar fashion to a typical forearm brace, of which general construction including use of Velcro straps and embroidery methods will not be described in detail since such information is generally well known and readily available. Brace 100 comprises two identical metal hooks 101, a tri-glide slide #1 102 (bottom/innermost) that is identical to a tri-glide slide #2 103 (top/outermost), a dumbbell strap 104 (polypropylene webbing), a wide Velcro strip 105 that collaborates with the dumbbell strap 104, three identical forearm straps 106 (nylon webbing) with identical Velcro strips 107 (one on each) on the underside, a male/female buckle 109, and a buckle strap 110 (polypropylene webbing). As visual aids to demonstrate the purpose of the device, an exemplary dumbbell weight 113, an exemplary human hand 114, and an exemplary human arm 115 are shown in this and other drawings. The components of the device as well as their purposes remain consistent throughout each drawing, as there is only one embodiment which is shown from multiple perspectives. Each drawing does not necessarily show all elements. Identical components are assigned the same reference number to simplify and reduce clutter in the drawings.

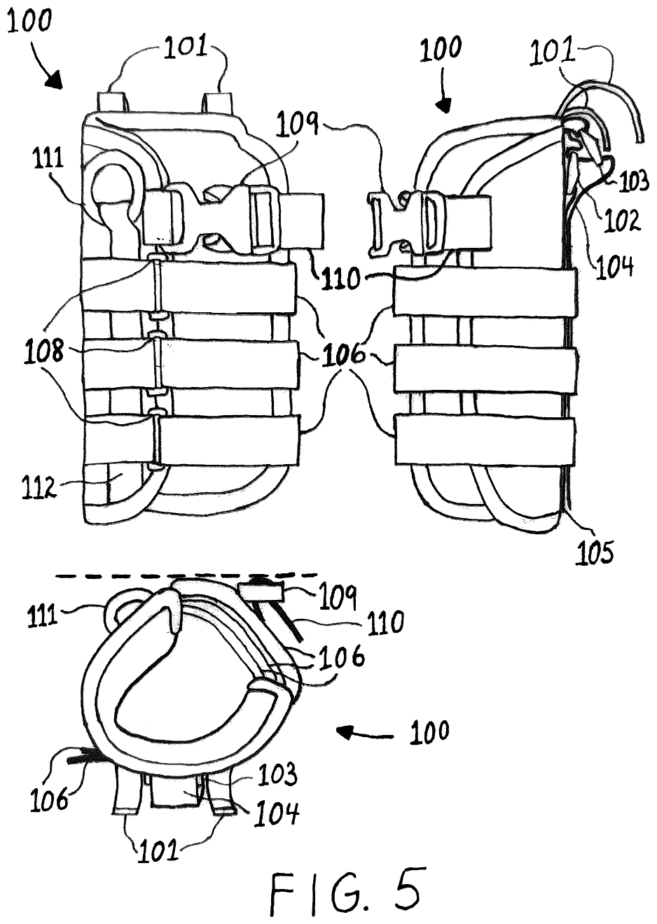

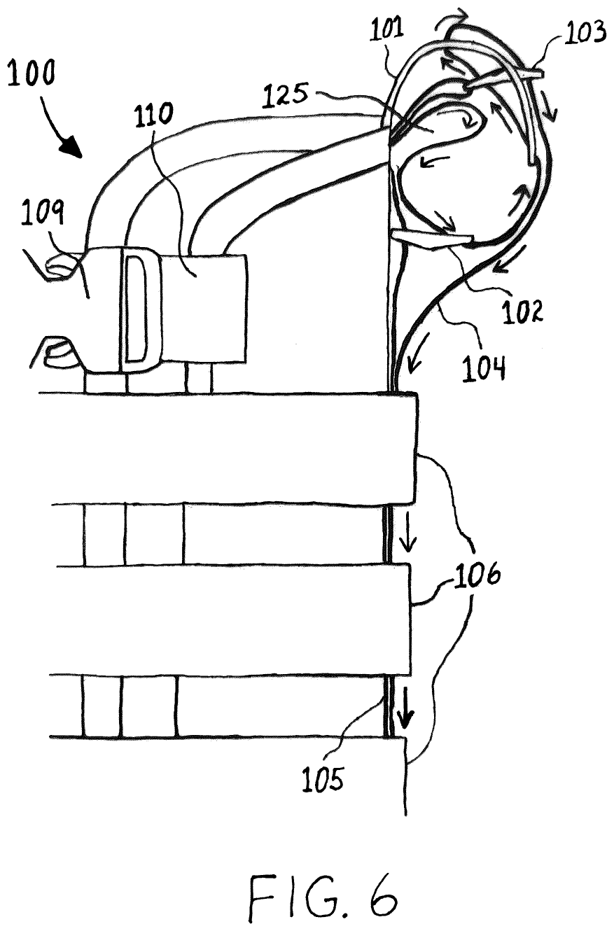

In this embodiment the brace 100 is worn by human arm 115 and rests at the base of human hand 114. The metal hooks 101 supply a contoured foothold for a dumbbell weight 113 to rest upon. Dumbbell strap 104 wraps over the dumbbell 113 and through tri-glide slide #1 102, which pulls the dumbbell 113 flush with the brace 100. Dumbbell strap 104 is then wrapped back over dumbbell weight 113 and through tri-glide slide #2 103, which pulls the dumbbell weight 113 down flush into the belly of the metal hooks 101. Dumbbell strap 104 is then wrapped once more over dumbbell weight 113 and pulled/pressed down onto wide Velcro strip 105 which secures the dumbbell weight 113 into place on the brace 100 at the base of the human hand 114, providing a realistic balance, feel, and weight distribution for the user. Forearm straps 106 are then wrapped around brace 100 and fastened down upon dumbbell strap 104 (which adheres to Velcro) and wide Velcro strip 105 using Velcro strips 107 which further secures dumbbell weight 113 and provides additional support. Buckle 109 and buckle strap 110 are used to provide increased stabilization to the device (in addition to forearm straps 106) and will be discussed further in FIG. 5. A close-up directional drawing of the flow of dumbbell strap 104 over dumbbell weight 113 and through tri-glide slide #1 102 and tri-glide slide #2 103 is provided as FIG. 6.

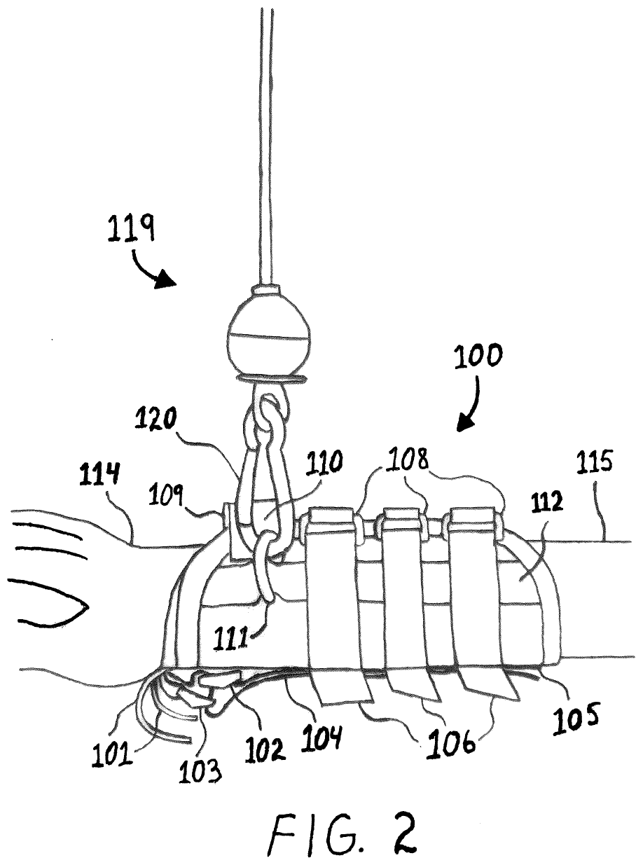

FIG. 2 illustrates the secondary purpose of the device as the embodiment of brace 100 in a left side view from a functional perspective for use with a cable weight machine 119. In this embodiment brace 100 is worn by human arm 115 and rests at the base of human hand 114 in the same fashion depicted in FIG. 1. O-ring strap 112 (polypropylene webbing) secures O-ring 111 to brace 100 which provides a fastening point for a cable weight machine 119 to attach its carabiner 120 to. This effectively anchors the weight from the cable machine 119 to the brace 100 at the base of the human hand 114, providing a realistic balance, feel, and weight distribution for the user. Three identical Uni-loops 108 are used to anchor and route forearm straps 106 in the same fashion that a standard forearm brace does. Previously disclosed elements from FIG. 1 are shown such as metal hooks 101, tri-glide slide #1 102, tri-glide slide #2 103, dumbbell strap 104, wide Velcro strip 105, forearm straps 106, buckle 109, and buckle strap 110.

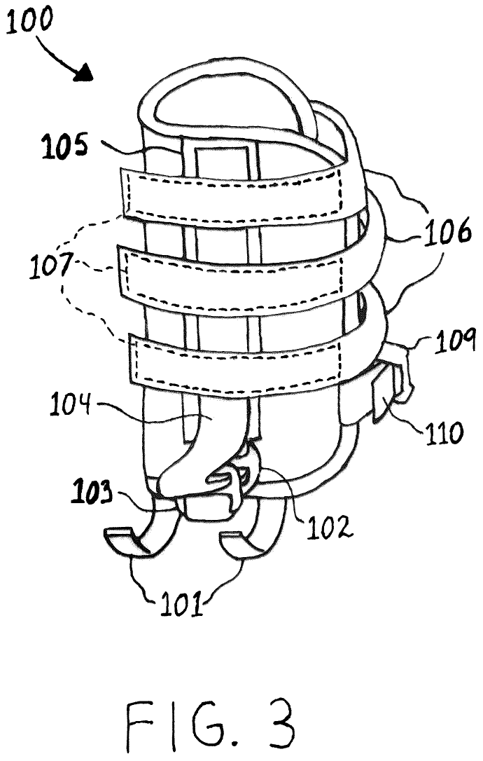

FIG. 3 provides an unobstructed view of the device from the same perspective as FIG. 1 for increased clarity and understanding. Brace 100 is shown without the presence of any exemplary elements so that certain parts of the device which were blocked from view in FIG. 1 are now visible in FIG. 3. Previously disclosed elements from FIG. 1 are shown, such as metal hooks 101, tri-glide slide #1 102, tri-glide slide #2 103, dumbbell strap 104, wide Velcro strip 105, forearm straps 106, Velcro strips 107, buckle 109, and buckle strap 110.

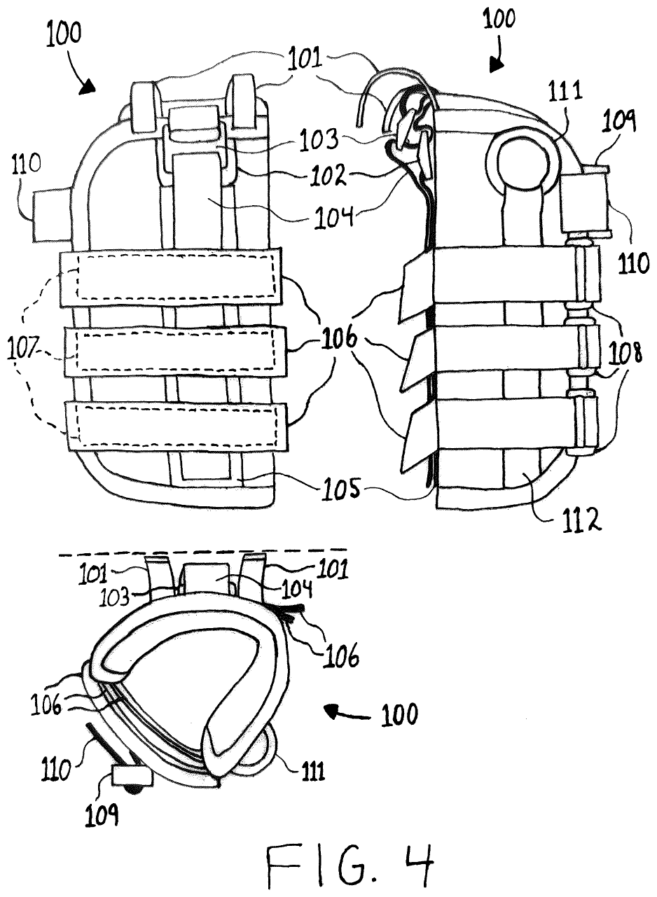

FIG. 4 is an isometric view of the device from a supinated perspective which also features a left side perspective and “down the barrel” perspective with metal hooks 101 at the far end of view (which exhibits the interior of brace 100). The primary purpose of this drawing is to show the features and components of the brace 100 from as many angles as possible from a supinated perspective. Previously disclosed elements from FIG. 1 and FIG. 2 are shown, such as metal hooks 101, tri-glide slide #1 102, tri-glide slide #2 103, dumbbell strap 104, wide Velcro strip 105, forearm straps 106, Velcro strips 107, Uni-loops 108, buckle 109, buckle strap 110, O-ring 111, and O-ring strap 112.

FIG. 5 is an isometric view of the device from a pronated perspective which also features a right side perspective and “down the barrel” perspective with metal hooks 101 at the far end of view which exhibits the interior of brace 100 in an inverse position as compared to FIG. 4. The pronated perspective exhibits how buckle 109 and buckle strap 110 are used to bridge the gap in brace 100 toward the opening near metal hooks 101, further reinforcing the hold supplied by forearm straps 106 on the user. The primary purpose of this drawing is to show the features and components of the brace 100 from as many angles as possible from a pronated perspective. Previously disclosed elements from FIG. 1 and FIG. 2 are shown, such as metal hooks 101, tri-glide slide #1 102, tri-glide slide #2 103, dumbbell strap 104, wide Velcro strip 105, forearm straps 106, Uni-loops 108, O-ring 111, and O-ring strap 112.

FIG. 6 is a close-up, partial-view, directional drawing of brace 100 depicting the flow of dumbbell strap 104 through tri-glide slide #1 102 and tri-glide slide #2 103. The starting point 125 shows where the process begins as well as where the dumbbell weight 113 (shown in FIG. 1) would be located during use of device at the base of metal hooks 101 and underneath dumbbell strap 104, flush with brace 100. Arrows indicate the way the dumbbell strap 104 is first woven through tri glide slide #1 102 then secondly back through tri glide slide #2 103 then finally back onto wide Velcro strip 105. It should be reiterated that dumbbell strap 104 begins at the base of metal hooks 101 and first travels over the handle of the dumbbell weight 113 (shown in FIG. 1) before weaving through tri glide slide #1 102. Dumbbell weight 113 (shown in FIG. 1) is not depicted in this drawing because it obscures the view of the directional flow of dumbbell strap 104, which is the primary purpose of FIG. 6. Previously disclosed elements from FIG. 1 are shown, such as forearm straps 106, buckle 109, and buckle strap 110.

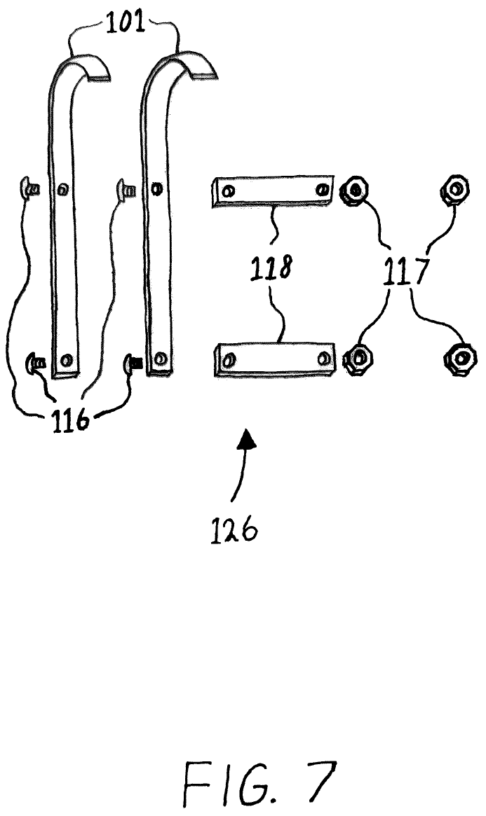

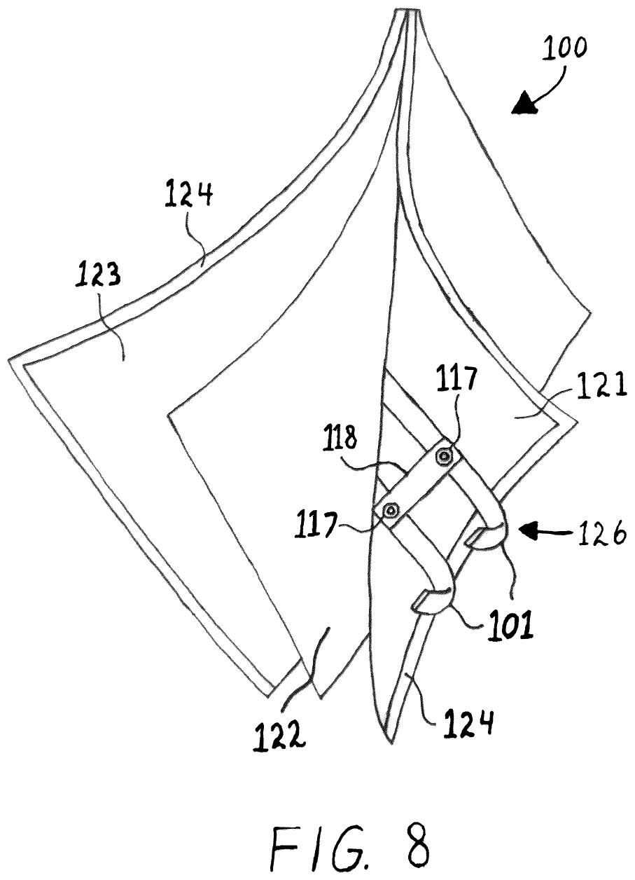

FIG. 7 illustrates an exploded view of the hook system 126 which supplies a contoured foothold for a dumbbell weight 113 (shown in FIG. 1) to rest upon. The hook system 126 is comprised of metal hooks 101 and additional elements. Metal hooks 101 are attached in a parallel fashion via two identical metal crossbars 118, which are secured with four identical screws 116 and their four identical respective nuts 117. This stable design eliminates any potential wiggle movement of metal hooks 101 during use of device. The hook system 126 is installed into brace 100—this is depicted in FIG. 8.

FIG. 8 illustrates an exploded view of the layers of material used in the construction of brace 100. The innermost layer of material which contacts human arm 115 (shown in FIG. 1 and FIG. 2) during use of device is neoprene 121 (1 mm), which is soft and comfortable to the touch. Plastic insert 122 (8 mm) provides a firm and durable quality and lays upon neoprene 121 (1 mm). The outermost layer of material, which houses the other components and contacts dumbbell weight 113 (shown in FIG. 1) during use of device, is 600 denier 123, which is strong and durable. The hook system 126 (shown here in assembled form as a partial view of metal hooks 101, one of the metal crossbars 118, and two of the screws 116) is placed in between neoprene 121 and plastic insert 122 and thoroughly secured to all three layers of material via embroidery. Embroidery methods will not be described in detail because such information is widely available, known, and understood. Two identical nylon webbing strips 124 are sewn over the edges of the material layers, one from each side, to completely seal the layers together which forms the foundation of brace 100, upon which all other elements described above are attached.

The various aspects of what is described above can be used alone or in various combinations. The teaching of the present application has numerous advantages. Different embodiments or implementations may yield one or more of the following advantages. It should be noted that this is not an exhaustive list and there may be other advantages which are not described herein. For example, one advantage of the teaching of this application is it provides for a device that grips a dumbbell weight in lieu of a hand. It is another advantage of the present invention that it provides for a device that grips a cable machine weight in lieu of a hand. It is yet another advantage of the present application that it allows a user to circumnavigate various different upper extremity injuries, deformities, and amputations in the context of weightlifting.

Although the teaching of the present application has been described in detail for purpose of illustration, it is understood that such detail is solely for that purpose, and variations can be made therein by those skilled in the art without departing from the scope of the teaching of this application. For example, the device has been described with three forearm straps, but it is understood that the device does not need to have three forearm straps, any other number of straps properly sized and balanced could suffice. Additionally, the use of straps to secure the device to the user is not mandatory, any other tightening and securing method used in arm braces and the like could suffice. Further, the device has been described with a hook system (comprised of two hooks) and an O-ring, but it should be understood that the teaching can also be applied to devices that have any number of hooks, curvatures, contours, or otherwise that supply a foothold for a dumbbell weight to rest upon as well as many other variations of fastening points for a cable machine carabiner to attach to. Additionally, the use of a dumbbell weight or cable machine is not mandatory, many other variations of weights could be used in conjunction with the teaching of this application to provide the advantages described herein. Another example is that the device has been described with a dumbbell strap that wraps around dumbbell weight which secures the weight to the brace, but it should be understood that the device does not need to have a dumbbell strap, many other methods of securing one object to another could be used, including but not limited to racks, tracks, slides, pins, loops, knots, etc. It should be noted that there are many alternative ways of implementing the methods and apparatuses of the teachings of the present application. Features described in the preceding description may be used in combinations other than the combinations explicitly described.

Whilst endeavoring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

The term “comprising” as used in the claims does not exclude other elements or steps. The term “a” or “an” as used in the claims does not exclude a plurality. A unit or other means may fulfill the functions of several units or means recited in the claims.

1. A device comprising a customized forearm brace wherein the brace can be attached to and effectively hold a weight, dumbbell or otherwise, in lieu of a hand, providing grip for the user who is unable to use a hand in the context of weightlifting.

2. A device according to claim 1, wherein a hook, hooks, or hook-shaped curvature affixed to the brace supply a foothold for a dumbbell weight to rest upon and a strap or straps wrap(s) around the dumbbell which secures the weight to the brace.

3. A device according to claim 2, wherein a ring, clip, carabiner, or related hardware affixed to the brace allows for the brace to be clipped onto or attached to a cable weightlifting machine (wherein the cable weightlifting machine replaces the use of the dumbbell weight).
